SEQUENCE LISTING (102 sequences)

SEQ ID NO: 1 - protein, 4 aa, B. subtilis
NHFL

SEQ ID NO: 2 - DNA, 21 bp, B. subtilis
aatcattttttgattaagccg

SEQ ID NO: 3 - DNA, 21 bp, B. subtilis
aatcattttttgaggtctccg

SEQ ID NO: 4 - DNA, 21 bp, B. subtilis
aatcattttctgcctcgttgg

SEQ ID NO: 5 - DNA, 21 bp, B. subtilis
aatcattttcttcctaaggtg

SEQ ID NO: 6 - DNA, 21 bp, B. subtilis
aatcattttctgttgccgccg

SEQ ID NO: 7 - DNA, 21 bp, B. subtilis
aatcattttttgcctcctcgg

SEQ ID NO: 8 - DNA, 21 bp, B. subtilis
aatcattttctgcctactggg

SEQ ID NO: 9 - DNA, 21 bp, B. subtilis
aatcattttctgatgccgaag

SEQ ID NO: 10 - DNA, 21 bp, B. subtilis
aatcattttcttaaggggacg

SEQ ID NO: 11 - DNA, 21 bp, B. subtilis
aatcattttttgccgcagaat

SEQ ID NO: 12 - DNA, 21 bp, B. subtilis
aatcattttcttctttggcgt

SEQ ID NO: 13 - DNA, 21 bp, B. subtilis
aatcattttctgattaggaag

SEQ ID NO: 14 - DNA, 21 bp, B. subtilis
aatcattttctgccgactgct

SEQ ID NO: 15 - protein, 7 aa, B. subtilis
NHFLPKV

SEQ ID NO: 16 - protein, 14 aa, B. subtilis
MVQKRNHFLPYSLP

SEQ ID NO: 17 - protein, 11 aa, B. subtilis
LFNKHVPGGGC

SEQ ID NO: 18 - protein, 12 aa, B. subtilis
NHFLNRPAQSQV

SEQ ID NO: 19 - protein, 7 aa, B. subtilis
NHFLIKP

SEQ ID NO: 20 - protein, 7 aa, B. subtilis
NHFLRSP

SEQ ID NO: 21 - protein, 7 aa, B. subtilis
NHFLPRW

SEQ ID NO: 22 - protein, 7 aa, B. subtilis
NHFLPKV

SEQ ID NO: 23 - protein, 7 aa, B. subtilis
NHFLLPP

SEQ ID NO: 24 - protein, 7 aa, B. subtilis
NHFLPPR

SEQ ID NO: 25 - protein, 7 aa, B. subtilis
NHFLPTG

SEQ ID NO: 26 - protein, 7 aa, B. subtilis
NHFLMPK

SEQ ID NO: 27 - protein, 7 aa, B. subtilis
NHFLKGT

SEQ ID NO: 28 - protein, 7 aa, B. subtilis
NHFLPQN

SEQ ID NO: 29 - protein, 7 aa, B. subtilis
NHFLLWR

SEQ ID NO: 30 - protein, 7 aa, B. subtilis
NHFLIRK

SEQ ID NO: 31 - protein, 7 aa, B. subtilis
NHFLPTA

SEQ ID NO: 32 - protein, 12 aa, B. subtilis
NHFLPPKMGPTD

SEQ ID NO: 33 - protein, 12 aa, B. subtilis
NHFLPEPRLVMP

SEQ ID NO: 34 - protein, 12 aa, B. subtilis
NHFLAPQPPVKP

SEQ ID NO: 35 - protein, 12 aa, B. subtilis
NHFLMPNPLLAM

SEQ ID NO: 36 - protein, 12 aa, B. subtilis
NHFLIPPEPLRE

SEQ ID NO: 37 - protein, 12 aa, B. subtilis
NHFLPLNPPAPS

SEQ ID NO: 38 - protein, 4 aa, B. subtilis
GGGC

SEQ ID NO: 39 - protein, 5 aa, B. subtilis
NHFLP

SEQ ID NO: 40 - protein, 7 aa, B. anthracis
TSQNVRT

SEQ ID NO: 41 - protein, 7 aa, B. anthracis
TYPIPIR

SEQ ID NO: 42 - protein, 7 aa, B. anthracis
TYPIPFR

SEQ ID NO: 43 - protein, 7 aa, B. anthracis
TYPVPHR

SEQ ID NO: 44 - protein, 4 aa, B. anthracis
TSQN

SEQ ID NO: 45 - protein, 7 aa, B. cereus
VTSRGNV

SEQ ID NO: 46 - DNA, 21 bp, B. cereus
acgcatcgtttgccttctgcg

SEQ ID NO: 47 - DNA, 21 bp, B. cereus
gttactagtgaggggaatgtt

SEQ ID NO: 48 - DNA, 21 bp, B. cereus
gaactgtgggtgattcctgca

SEQ ID NO: 49 - DNA, 21 bp, B. cereus
tattcgcctcctcatgagcat

SEQ ID NO: 50 - protein, 7 aa, B. anthracis
NSVTLEP

SEQ ID NO: 51 - protein, 7 aa, B. anthracis
KPRQPGL

SEQ ID NO: 52 - protein, 7 aa, B. anthracis
STPAWLS

SEQ ID NO: 53 - protein, 7 aa, B. anthracis
SYPHGQI

SEQ ID NO: 54 - protein, 7 aa, B. anthracis
FTGTLNP

SEQ ID NO: 55 - protein, 7 aa, B. anthracis
RTPSLSP

SEQ ID NO: 56 - protein, 7 aa, B. anthracis
FSVPRMP

SEQ ID NO: 57 - DNA, 21 bp, B. anthracis
tcgtatcttccgtattttgat

SEQ ID NO: 58 - DNA, 21 bp, B. anthracis
cttttgtcgcctctgcatcgt

SEQ ID NO: 59 - DNA, 21 bp, B. anthracis
ttttgatctccgcttcgtgcg

SEQ ID NO: 60 - DNA, 21 bp, B. anthracis
aatagtgttactcttggaccg

SEQ ID NO: 61 - DNA, 21 bp, B. anthracis
gaaccggaggcaccgggtttg

SEQ ID NO: 62 - DNA, 21 bp, B. anthracis
tctactccgggctggctgtcg

SEQ ID NO: 63 - DNA, 21 bp, B. anthracis
actagtcagaatgtgcggacg

SEQ ID NO: 64 - DNA, 21 bp, B. anthracis
acttatcctattccgaatcgt

SEQ ID NO: 65 - DNA, 21 bp, B. anthracis
acttatcctaatccgtttcgt

SEQ ID NO: 66 - DNA, 21 bp, B. anthracis
tcttatcctcatggtcagatt

SEQ ID NO: 67 - DNA, 21 bp, B. anthracis
tttactgggactcttaatcct

SEQ ID NO: 68 - DNA, 21 bp, B. anthracis
acttatccggtgccgcatcgg

SEQ ID NO: 69 - DNA, 21 bp, B. anthracis
cggactccttcgcttcctagt

SEQ ID NO: 70 - DNA, 21 bp, B. anthracis
tttagtgttcctcgtatgccg

SEQ ID NO: 71 - DNA, 21 bp, B. cereus
tggtcgccgctgcatgaacat

SEQ ID NO: 72 - protein, 7 aa, B. cereus
THRLPSR

SEQ ID NO: 73 - protein, 7 aa, B. cereus
VTSRGNV

SEQ ID NO: 74 - protein, 7 aa, B. cereus
KLWVIPQ

SEQ ID NO: 75 - protein, 7 aa, B. cereus
YSPLHRH

SEQ ID NO: 76 - protein, 7 aa, B. cereus
SYPPYFD

SEQ ID NO: 77 - protein, 7 aa, B. cereus
LLSPLHR

SEQ ID NO: 78 - protein, 7 aa, B. cereus
FDSPLRR

SEQ ID NO: 79 - protein, 7 aa, B. cereus
WSPLHKH

SEQ ID NO: 80 - protein, 12 aa, B. cereus
NHFLKSQPGVVT

SEQ ID NO: 81 - protein, 6 aa, B. cereus
  VARIANT 4: Xaa = His or Arg
  VARIANT 5: Xaa = Arg or Lys
SPLXXH

SEQ ID NO: 82 - protein, 6 aa, B. cereus
  VARIANT 4: Xaa = His or Arg
  VARIANT 5: Xaa = Arg or Lys
SPLXXH

SEQ ID NO: 83 - protein, 4 aa, B. subtilis
NHFL

SEQ ID NO: 84 - protein, 7 aa, B. subtilis
  VARIANT 4: Xaa = Val, Ile
  VARIANT 6: Xaa = Ile, Phe, His
TYPXPXR

SEQ ID NO: 85 - protein, 7 aa, Artificial Sequence
  TYP consensus sequence
  VARIANT 4: Xaa =  Val, Ile, Pro, Leu,
  VARIANT 6: Xaa = Ile, Phe, His, Thr
TYPXPXR

SEQ ID NO: 86 - protein, 7 aa, Artificial Sequence
  TYP sequence
TYPLPIR

SEQ ID NO: 87 - protein, 7 aa, B. anthracis
TYPLPIR

SEQ ID NO: 88 - protein, 7 aa, B. anthracis
TYPPPIR

SEQ ID NO: 89 - protein, 11 aa, Bacillus
TYPLPIRGGGC

SEQ ID NO: 90 - protein, 11 aa, Bacillus
HWHHHGHGGGC

SEQ ID NO: 91 - protein, 11 aa, Bacillus
ILPRPYTGGGC

SEQ ID NO: 92 - protein, 8 aa, B. subtilis
ATYPLPIR

SEQ ID NO: 93 - protein, 12 aa, B. anthracis
ATYPLPIRGGGC

SEQ ID NO: 94 - protein, 8 aa, B. anthracis
  VARIANT 5: Xaa = Val, Ile Pro, Leu
  VARIANT 7: Xaa = Ile, Phe, His, Thr
ATYPXPXR

SEQ ID NO: 95 - protein, 6 aa, B. anthracis
SLLPGL

SEQ ID NO: 96 - protein, 11 aa, B. anthracis
SLLPGLPGGGC

SEQ ID NO: 97 - protein, 12 aa, B. anthracis
  VARIANT 5: Xaa = Val, Ile, Pro, Leu
  VARIANT 7: Xaa = Ile, Phe, His, Thr
ATYPXPXRGGGC

SEQ ID NO: 98 - DNA, 21 bp, B. anthracis
acttatccgccgccgactctt

SEQ ID NO: 99 - protein, 6 aa, B. anthracis
SPLNNH

SEQ ID NO: 100 - protein, 11 aa, B. anthracis
NHFLPKVGGGC

SEQ ID NO: 101 - DNA, 21 bp, B. anthracis
acgtatccgctgccgattccg

SEQ ID NO: 102 - DNA, 21 bp, B. anthracis
acttatccgctgcctattagg

(19) United States
(12) Patent Application Publication
(10) Pub. No.: US 2007/0281302 A1
(43) Pub. Date: Dec. 6, 2007

(54) Peptide ligands for Bacillus anthracis spore detection

(76) Inventor: Charles L. Turnbough JR., Vestavia Hills, AL (US)

Correspondence Address: Charles L. Turnbough, Jr., 2312 Overlook Crest, Vestavia Hills, AL, 35226, US

(21) Appl. No.: 11/216,002

(22) Filed: Sep. 1, 2005

Related U.S. Application Data
(63) Continuation of application No. 10/954,483, filed on Oct. 1, 2004, now abandoned.
Application No. 10/954,483 is a continuation-in-part of application No. 09/229,751, filed on Jan. 14, 1999, now abandoned.
(60) Provisional application No. 60/071,411, filed on Jan. 14, 1998.

(51) Int. Cl. C12Q 1/68; G01N 33/554; C07K 14/32

(52) U.S. Cl. 435/6; 435/7.32; 530/350

(57) ABSTRACT

The present invention relates to peptides that bind to the bacterial spores, such as B. anthracis, B. subtilis and B. cereus spores. The present invention also relates to method of identifying such peptides using phage display ligand screening system. The present invention further relates to the use of such peptides for the detection of bacterial spores, such as Bacillus anthracis spores.

This application is a continuation-in-part of U.S. patent application Ser. No. 09/229,751, filed Jan. 14, 1999, which claims priority from U.S. Provisional Application Ser. No. 60/071,411, filed Jan. 14, 1998. The entirety of these applications are incorporated herein by reference.

### FIELD OF THE INVENTION

The present invention relates to means of identifying and capturing spore-forming bacteria comprising preparation of peptides which bind to the surfaces of bacterial spores. The peptides are identified using a phage display ligand screening system.

### BACKGROUND OF THE INVENTION

The capture and identification of bacterial spores is useful for detecting pathogenic or otherwise harmful bacteria. Often the presence of spores can indicate to the researcher or epidemiologist the presence of virulent organisms. It is also important to determine the presence of spores of pathogenic organisms in the environment in order to more effectively control spread of infections. The ability to produce a monitorable tag or ligand that will bind specifically to the bacterial spore would provide a valuable tool for identifying pathogenic organisms in the infected patient and in the environment. The bacterial-specific tag or ligand may also find important applications in the ongoing war against bioterriorism.

For example, the gram-positive soil bacterium B. anthracis, the causative agent of anthrax, has been developed into a weapon of mass destruction by numerous terrorist groups [(Inglesby et al., JAMA 281:1735-1745 (1999)]. B. anthracis is an effective agent for biological warfare and terrorism primarily because it forms spores. Spores are resistant to extreme temperatures, noxious chemicals, desiccation, and physical damage, which makes them suitable for incorporation into explosive weapons and for concealment in terrorist devices [(Nicholson et al., Microbiol. Mol. Biol. Rev. 64:548-572 (2000)]. Spores enter the body through skin abrasions or by ingestion or inhalation. Once exposed to internal tissues, the spores germinate and vegetative cell growth ensues, often resulting in the death of the host within several days [Inglesby, JAMA 287:2236-2252 (2002); Swartz, N. Engl. J. Med. 345:1621-1626 (2001)]. Natural strains of B. anthracis are sensitive to common antibiotics that can be used to treat anthrax. However, to ensure a successful outcome, treatment must begin within a day or two after exposure to spores [(Jernigan, et al., Emerg. Infect. Dis. 7:933-944 (2001)]. Thus, rapid detection of B. anthracis spores is critical in responding to the anthrax threat.

Several detection systems are currently used to identify B. anthracis. The most accurate systems employ either PCR-based assays or traditional phenotyping of cultured bacteria [Bell et al., J. Clin. Microbiol. 40:2897-2902 (2002); Higgins et al., Appl. Environ. Microbiol. 69:593-599 (2003); Swartz N. Engl. J. Med. 345:1621-1626 (2001)]. However, these methods are complex, expensive, cumbersome, and slow, typically requiring spore germination and outgrowth of vegetative cells. Other systems, less complex and more portable, are based on antibody binding to spore surface antigens. These systems are relatively fast because they detect spores directly. However, current antibody-based detectors suffer from a lack of accuracy and limited sensitivity, which result in an unacceptably high level of both false-positive and false-negative responses, according to federal government trials (www.gsa/gov/mailpolicy) and other independent tests [D. King, et al., J. Clin. Microbiol. 41:3454-3455 (2003)]. The lack of accuracy with these systems is compounded by the normal presence in the environment of Bacillus spores that resemble (and share surface antigens with) B. anthracis spores. Particularly problematic are spores of the opportunistic human pathogen B. cereus and the insect pathogen B. thuringiensis, species which, based on genome sequence comparisons, are the most similar to B. anthracis [Read et al., Nature, 423:81-86 (2003)]. These three species, along with B. mycoides, comprise the phylogenetically similar B. cereus group [(Ash et al., Lett. Appl. Microbiol. 13:202-206 (1991); Priest, F. G. Systematics and ecology of Bacillus, p. 3-16. In A. L. Sonenshein, J. A. Hoch, and R. Losick (ed.), Bacillus subtilis and other gram-positive bacteria. Biochemistry physiology and molecular biology, American Society for Microbiology, Washington, D.C. (1993)]. Therefore, due to the aforementioned limitations and deficiencies, all currently available systems for detecting B. anthracis are inadequate for frontline use by emergency workers and soldiers on the battlefield and for routine monitoring of public areas. Clearly, there is an urgent need, for a better detector that can be used where the threat of B. anthracis spore exposure is the greatest.

The use of phage-displayed peptide libraries to identify peptide sequences that will bind to particular receptors has been used to evaluate the structure of proteins. [(See D'Mello, et al., Virology, 237(2): 319-26 (1997) and Salonen, et al., J. General Virology, 79 (pt4): 659-65 (1998) regarding mapping of antibodies and Marzari, et al., FEBS Lett. 411(1): 27-31 (1997) regarding phage display of B. thuringiensis insecticidal toxin]. A synopsis regarding the use of phage display has been reviewed by Smith and Petrenko in Chemical Reviews, 97(2): 391-410 (1997). However, none of the above references suggest using phage display peptide libraries for identifying peptide sequences which bind to whole cells, such as bacteria spores.

### SUMMARY OF THE INVENTION

One aspect of the present invention relates to the peptides that bind specifically to microorganisms and their spores, the compositions comprising the peptides, and the bacteria detection kits comprising the peptides or the compositions.

In one embodiment, an isolated peptide binds specifically to B. anthracis, said peptide is selected from the formula set forth in ATYPX₃PX₄R (SEQ ID NO: 129) and ATYPX₃PX₄RGGGC (SEQ ID NO:132), wherein said X₃ is a Ile, Val, Leu or P; X₄ is Ile, Phe, His, or Thr. Preferably, X₃ is a Leu and X₄ is Ile.

In another embodiment, an isolated peptide which binds to a B. anthracis, said peptide comprising an amino acid sequence of SEQ ID NO: 50, SEQ ID NO: 51, SEQ ID NO: 52, SEQ ID NO: 40, SEQ ID NO: 41, SEQ ID NO: 42, SEQ ID NO: 53, SEQ ID NO: 54, SEQ ID NO: 43, SEQ ID NO: 55, SEQ ID NO: 56, SEQ ID NO: 121, SEQ ID NO: 122, or SEQ ID NO: 123. Preferably, the peptide has an amino acid sequence of SEQ ID NO: 40, SEQ ID NO: 41, SEQ ID NO: 42, SEQ ID NO: 121, SEQ ID NO: 122, or SEQ ID NO: 123.

In another embodiment, an isolated peptide which binds to B. subtilis comprising an amino acid sequence of Asn-His-Phe-Leu (NHFL) (SEQ ID No. 1). Additional amino acid containing proline, to provide a sequence NHFLP (SEQ ID No. 39) is particularly preferred sequence.

Yet, in another embodiment, an isolated peptide which binds to B. cereus comprising sequences Val-Thr-Ser-Arg-Gly-Asn-Val (VTSRGNV) (SEQ ID No. 100). Another embodiment, an isolated peptide which binds to B. cereus comprising a sequence of Ser-Pro-Leu-X₁-X₂-His, wherein X₁ is His or Arg and X₂ is Arg or Lys (SPLX₁X₂H).

Another aspect of the present invention relates to methods for identifying peptides that bind specifically to microorganisms, such as B. anthracis, B. subtilis, and B. cereus. Briefly, random peptides are generated in phage display peptide libraries and the peptides that specifically bind to microorganisms are identified using biopanning.

In one embodiment, the random peptides are generated by commercially available phage display peptide libraries. In another embodiment, the microorganisms are bacteria spores.

Another aspect of the present invention relates to methods of capture and/or detecting of microorganisms and/or their spores in an environmental, clinical, and anti-bioterrorism setting, using microorganism-specific peptides identified by the methods of the present invention.

In one embodiment, the microorganism-specific peptides are coupled to detectable (e.g., fluorescent, phosphorescent, radioactive, etc.) tags and the peptide-tag conjugates are mixed with a sample which contains cognate spores.

Yet another aspect of the present invention relates to methods for protecting potential hosts from exposure to disease-causing spores by administration of peptides which bind to the spores.

### BRIEF DESCRIPTION OF THE FIGURES

FIG. 1 is a schematic drawing of biopanning a phage display peptide library for phage/peptides that bind spores.

FIG. 2 depicts a representative FACS analysis of the binding of an NH-peptide-phycoerythrin conjugate to B. subtilis 168 spores.

FIG. 3 is FACS analysis of the binding of the TYPLPIRGGGC-PE conjugate to selected Bacillus spores.

FIG. 4 is FACS analysis comparing the abilities of the TYPLPIRGGGC-PE (TYP peptide-PE) and ATYPLPIRGGGC-PE (ATYP peptide-PE) conjugates to bind to selected Bacillus spores. It also shows the binding results for a control peptide (HWHHHGHGGGC)-PE conjugate.

FIG. 5 is Fluorescence microscopy showing selective binding of ATYPLPIRGGGC-PE to B. anthracis spores.

FIG. 6 is FACS analysis contrasting the binding of the ATYPLPIRGGGC-PE (ATYP peptide-PE) and SLLPGLPGGGC-PE (SLLPGL peptide-PE) conjugates to selected Bacillus spores. It also shows the binding results for a control peptide (HWHHHGHGGGC)-PE conjugate.

### DETAILED DESCRIPTION OF THE INVENTION

The primary objective of the present invention is to identify short peptide ligands that bind specifically to the spores of microorganisms, particularly those of Bacillus species, such as B. anthracis. The peptide ligands will bind tightly and in a species-specific manner to a physiological or fortuitous receptor on the surface of the spore.

Spores of primary interest in the present disclosure were spores of Bacillus anthracis, Bacillus subtilis, Bacillus cereus, and Bacillus thuringiensis. However, methods of the invention may also be used to identify and capture other pathogens such as Clostridia species. B. anthracis is a key target because of its potential as an agent for use in biological warfare and terrorism. B. subtilis is a target primarily because it is used as a stimulant in the development of detection devices for pathogenic B. anthracis. B. cereus and B. thuringiensis are targets because they closely resemble B. anthracis and because they are widely distributed in the environment. Thus, they can, potentially, produce false positive readings in detection devices and systems used to identify B. anthracis spores.

In one embodiment, phage display ligand screening is employed using combinatorial library of 2×10⁹ random 7-mer or 12-mer peptide sequences. The peptides are individually displayed on the surface of the filamentous coliphage M13. The random peptides are fused to the amino terminus of the minor coat protein PIII. The library is made by inserting a random nucleotide sequence at the beginning of the pIII gene of many copies of the M13 genome. These recombinant genomes are used to produce M13 phage. Each recombinant pIII gene produced a random peptide-pIII fusion protein and five copies of this fusion protein are displayed at one end of the mature phage particle. Thus, the random peptide sequence is displayed at the amino terminus of each pIII copy for a given phage. Furthermore, the random peptide sequence displayed by a particular phage clone can be readily determined by sequencing the peptide-encoding region of the phage genome.

The present invention provides a method comprises biopanning the phage display library by mixing phages from the phage display library with spores, incubating the mixture at about room temperature and separating the phage-spore complexes by centrifugation. The phage-spore complexes are washed several times in a buffer. The phages are eluted from the phage-spore complexes with cold buffer at low pH, and quickly neutralized to prevent phage killing. The phages can then be amplified by infecting an appropriate organism. (e.g., E. coli) The cell lysate obtained from the culture may optionally be subjected to the previous steps repeatedly. After several rounds of biopanning, (preferably about two to five rounds, more preferably, 4 rounds) individual clones are purified from the eluted phages. Phage plaques are amplified, the genomic DNA extracted and the DNA sequences of the 7-mer and 12-mer peptides encoding region determined. The DNA sequences indicate the sequences of the tight-binding peptides. The indicated peptide sequences are tagged and exposed to known spores to determine their binding properties.

Variations in the process would be known to one of skill in the art. Some of the modifications which enhance productivity are provided in the Examples described hereinafter. Modified versions of the biopanning procedure can also be used wherein phage are permitted to bind spores. Binding complexes are recovered by centrifugation. Complexes are mixed with E. coli to permit phage amplification (under conditions where B. subtilis growth is inhibited), and amplified phage are subjected to additional rounds of biopanning. Tight-binding phage are then recovered by centrifuging spores plus bound phage through a density gradient.

While the method of the present invention were first practiced targeting B. subtilis, then targeting B. anthracis, Bacillus thuringiensis and B. cereus, the methods disclosed herein, particularly the biopanning methods, may be used to identify useful sequences for binding to surfaces of other microorganisms.

The microorganism-specific peptides identified by the methods of the invention may be prepared by means known in the art. For example, the microorganism-specific peptides can be synthesized using solid-phase synthesis and standard F-MOC chemistry. The synthesized microorganism-specific peptides can be used in the detection/screening methods described herein and comparable methods known in the art.

Another objective of the present invention is to use microorganism-specific peptides to capture the cognate spore in filters or as part of a detection device (e.g., a capture device that concentrates the spores for identification by mass spectroscopy, DNA/RNA sequence evaluation, etc.). The microorganism-specific peptides can also be used directly in microorganism detection/identification devices and procedures. The microorganism-specific peptides can be coupled to detectable (e.g., fluorescent, phosphorescent, radioactive, etc.) tags and the peptide-tag conjugates mixed with a sample which may contain cognate spores. If spores are present, they will be bound by the peptide-tag, thereby marking the spores for detection by whatever detector is appropriate for the particular tag.

For example, when tagged sequences which bind to the surface of B. subtilis spores, particularly 5-mer to 12-mer sequences, are placed in the environment believed to contain B. anthracis spores, the presence of the bacteria of interest are identified. Tags such as fluorescent, phosphorescent or colorimetric tags make it possible to visualize the presence of the bacteria. Other tags, such as radioactive tags, may require other equipment such as scintillators to determine the presence or absence of the target organisms. The method described above is particularly useful for identifying contamination of water and food that might cause disease when ingested. Contamination of the air might be established using methods of the invention. The latter is particularly important when the possible contaminant is B. anthracis. It would be possible to attach the peptides identified as having the appropriate binding properties to solid supports to capture spores or spore-forming organisms which bind to the peptide. The particular support will depend on the use. For example, appropriate supports may be natural fibers or polymers which may be in the form of filtering devices, tapes or sponges. Supports having the binding peptides may be used as protective barriers such as masks.

Purified peptides formulated in pharmaceutically acceptable carriers such as buffered saline may be administered to animals in an appropriate amount to elicit an immune response or to bind to the spore to cause alteration in pathogenicity. The method of administration will depend on the organism and the site of infection. Formulations for inhalation may also be buffered to prevent damage to tissue.

The microorganism-specific peptides may also be used as antigens in the preparation of vaccines against pathogenic spore-forming bacteria. Polyclonal antibodies to the sequences of interest can be produced in animals and purified directly from the spleen cells. It is also common to isolate spleen cells from the animal for purposes of producing antibodies. These cells can then be fused with an immortal cell line and screened for monoclonal antibody secretion. Purified antibodies that specifically bind the peptide are within the scope of the present invention. The antibody can be labeled by means generally known in the art using, for example, fluorescent, radioactive or phosphorescent markers, or tags may be used in conjunction with a labeled secondary antibody in methods such as ELISA tests. Monovalent, divalent or single chain antibodies can be made which bind the peptides of the invention.

Anti-idiotype antibodies can also be made by means commonly known in the art. Antibodies to the present peptides can exhibit idiotypic mimicry and can be administered to provide protection against bacterial infection. Antibodies to the spore-binding peptides provided herein can be administered to susceptible hosts to block SpsC binding to the spore surface, thus inhibiting development of clinical disease.

The present invention is further illustrated by the following examples which should not be construed as limiting. The contents of all references, patents and published patent applications cited throughout this application, as well as the Figures and Tables are incorporated herein by reference.

### EXAMPLE 1

### General Method of Biopanning of Phase Display Peptide Libraries with Bacillus Spores

Peptides of interest were identified using a phage display ligand screening system. A phage display peptide library kit (New England Bio Labs) was used according to instructions of the manufacturer in the identification process. These libraries display random 7- and 12-mer peptides, respectively, on the surface of the filamentous coliphage M13. These peptides are fused to the surface-exposed amino terminus of the minor capsid protein pIII, which is present in five copies at one end of the phage particle. Thus, each phage displays five copies of one particular peptide. The phage display peptide libraries, each contain approximately 2×10⁹ independent phage clones. Each clone produces a protein pIII-peptide fusion that was created by the insertion of a random 21- or 36-base DNA fragment into a cloning site at the start of gene III of the phage. Consequently, the sequence of any peptide that binds a target spore can be readily determined by cloning the phage displaying this peptide and sequencing the peptide-encoding region of the phage genome. Spores were produced by cells grown in liquid Difco sporulation medium at 37° C. for 48-72 hours with shaking [Nicholson et al., Sporulation, germination and outgrowth. In: Harwood, C. R., Cutting, S. M. (Eds.), Molecular Biological Methods for Bacillus. Wiley, West Sussex, pp. 391-450, 1990)]. Spores were purified by sedimentation through a Renografin step gradient as previously described by Henriques et al., J. Bacteriol, 177: 3394-3406 (1995).

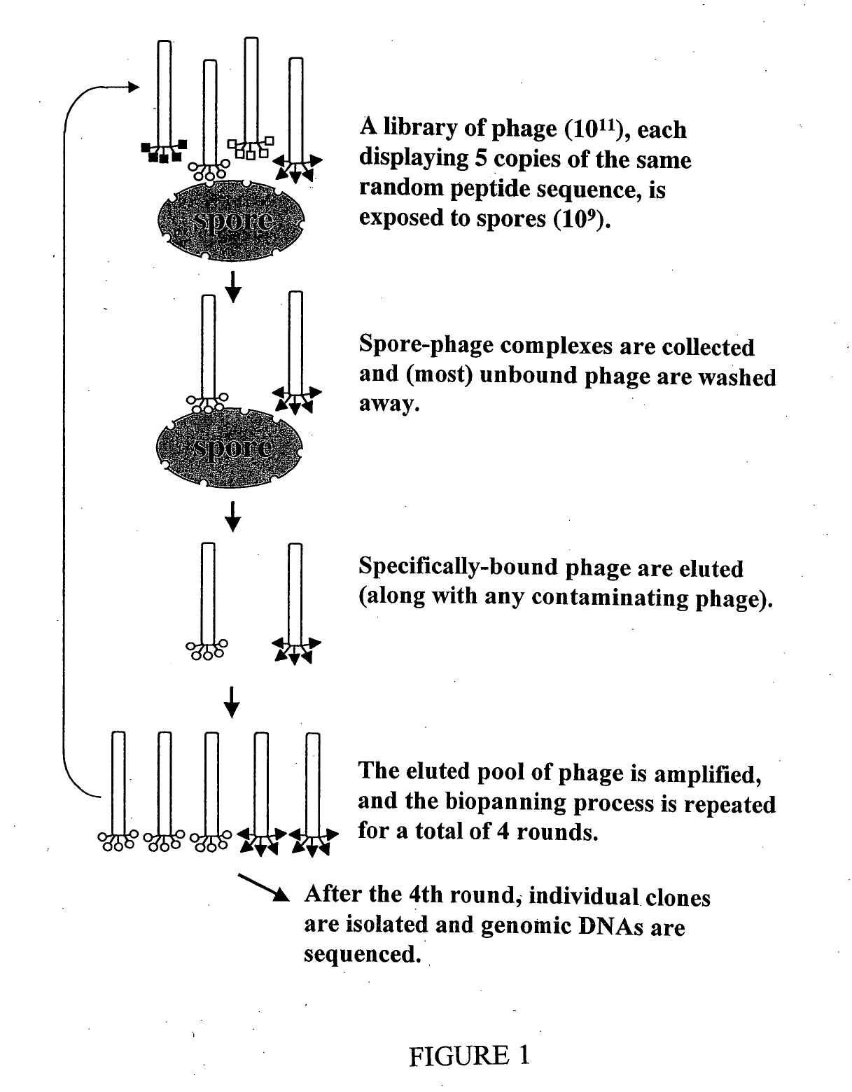

For a typical biopanning experiment, 10⁹ spores and 10¹¹ phage are mixed in 1 ml of sterile TBST [50 mM Tris-HCl (pH 7.5), 150 mM NaCl, 0.5% Tween-20] for approximately 20 min at room temperature with gentle shaking (FIG. 1). Spores and bound phage are collected by centrifugation (12,000×g at 4° C. for 5 min), and unbound phage in the supernatant are discarded. The pelleted spore-phage complexes are washed 10 times with 1 ml (each) of cold (4° C.) TBST to remove unbound phage. The pelleted spore-phage complexes are resuspended in 1 ml of elution buffer [0.2 mM glycine-HCl (pH 2.2), 1 mg/ml bovine serum albumin] and gently mixed for 5 min at room temperature. This sample is centrifuged as above for 5 min, and the supernatant, which contains eluted phage, is quickly removed and neutralized by the addition of 150 μl of 1 M Tris-HCl (pH 9.1) to prevent phage killing. The eluted phage are then amplified by infecting a phage-sensitive strain of E. coli (e.g., strain ER2738 from NEB). The resulting phage stock is used for a second round of biopanning, which is performed exactly as described above. A total of four rounds of biopanning are performed, after which the final eluted phage are plated to obtain single plaques. These plaques (typically around 30) are used to prepare phage stocks, from which genomic DNAs are extracted by using Qiagen Spin M13 columns. The sequence of the region of each genomic DNA encoding a putative spore-binding peptide is determined. The biopanning process is depicted in FIG. 1.

### EXAMPLE 2

### Identification of B. subtilis-Specific Peptides

(a) Determination of the 7-Mer Peptides

The DNA of over thirty independent phage isolates of B. subtilis were sequenced and thirteen unique sequences were identified. (See Table 1.) All encoded peptides contained amino terminal sequence Asn-His-Phe-Leu (SEQ ID No. 118). Although the sequences at positions five through seven are not identical, there is a clear preference for certain amino acids.

TABLE 1

Nucleotide and Amino Acid Sequences from
B. subtilis Spore-Binding Phage

| | |
|---|---|
| Isolate | |
| 1 AAT CAT TTT TTG ATT AAG CCG | (SEQ ID No. 2) |
| 2 AAT CAT TTT TTG AGG TCT CCG | (SEQ ID No. 3) |
| 3 AAT CAT TTT CTG CCT CGT TGG | (SEQ ID No. 4) |
| 4 AAT CAT TTT CTT CCT AAG GTG | (SEQ ID No. 5) |
| 5 AAT CAT TTT CTG TTG CCG CCG | (SEQ ID No. 6) |
| 6 AAT CAT TTT TTG CCT CCT CGG | (SEQ ID No. 7) |
| 7 AAT CAT TTT CTG CCT ACT GGG | (SEQ ID No. 8) |
| 8 AAT CAT TTT CTG ATG CCG AAG | (SEQ ID No. 9) |
| 9 AAT CAT TTT CTT AAG GGG ACG | (SEQ ID No. 10) |
| 10 AAT CAT TTT TTG CCG CAG AAT | (SEQ ID No. 11) |
| 11 AAT CAT TTT CTT CTT TGG CGT | (SEQ ID No. 12) |
| 12 AAT CAT TTT CTG ATT AGG AAG | (SEQ ID No. 13) |
| 13 AAT CAT TTT CTG CCG ACT GCT | (SEQ ID No. 14) |
| 1 Asn His Phe Leu Ile Lys Pro | (SEQ ID No. 19) |
| 2 Asn His Phe Leu Arg Ser Pro | (SEQ ID No. 20) |
| 3 Asn His Phe Leu Pro Arg Trp | (SEQ ID No. 21) |
| 4 Asn His Phe Leu Pro Lys Val | (SEQ ID No. 22) |
| 5 Asn His Phe Leu Leu Pro Pro | (SEQ ID No. 23) |
| 6 Asn His Phe Leu Pro Pro Arg | (SEQ ID No. 24) |
| 7 Asn His Phe Leu Pro Thr Gly | (SEQ ID No. 25) |
| 8 Asn His Phe Leu Met Pro Lys | (SEQ ID No. 26) |
| 9 Asn His Phe Leu Lys Gly Thr | (SEQ ID No. 27) |
| 10 Asn His Phe Leu Pro Gln Asn | (SEQ ID No. 28) |
| 11 Asn His Phe Leu Leu Trp Arg | (SEQ ID No. 29) |
| 12 Asn His Phe Leu Ile Arg Lys | (SEQ ID No. 30) |
| 13 Asn His Phe Leu Pro Thr Ala | (SEQ ID No. 31) |

For purposes of this application, discussions relating to the particular peptides will refer to the isolate numbers at the left side of the table. The SEQ ID No.'s relate to the computer-readable print-out which must be provided to the various patent offices.

To confirm that the B. subtilis peptides are tight-binding ligands, the following experiment was performed. 10⁷ phage of isolate #4 (NBFLPKV) (SEQ ID No. 15) and 10¹⁰ phage containing random 7-mer sequences were mixed with 10⁹ spores. This mixture was subjected to a single round of biopanning. The eluted phage were plaque-purified and genomic DNA was sequenced as described above. Seven of the ten phage examined contained the sequence of isolate #4. Thus, there was a 700-fold enrichment of this phage, clearly indicating that the isolate #4 peptide bound tightly to the spore.

Attempts to bind the spores of B. subtilis with the 4-mer peptide NHFL (SEQ ID No. 1) showed that sequence to be a poor ligand. However, the 5-mer sequence NHFLP (SEQ ID No. 39) showed tight binding.

In a search of the Swiss-Prot data base of characterized peptides for proteins containing the sequence NHFLP (SEQ ID No. 39), seven proteins with this sequence were identified. Five are eukaryotic proteins and two are B. subtilis proteins. The first B. subtilis protein is SpsC (Database accession number BG10611), which contains the NHFLP sequence near its amino terminus (i.e., MVQKRNHFLPYSLP-) (SEQ ID No. 16). SpsC appears to be involved in the synthesis of polysaccharides on the surface of the spore. It is probable that this protein uses its amino terminus to attach to a receptor on the spore surface. The instantly claimed peptide ligands may bind to the same site. The second B. subtilis protein is UvrC (Database accession number BG10349), an exonuclease involved in DNA repair. The NHFLP (SEQ ID No. 39) sequence is found in the middle of UvrC, which contains 598 amino acids. Because UvrC is known to be cytoplasmic, a connection between this protein and the peptide ligands is not obvious.

Alternatively, differential display can be utilized to quickly find small molecule analogs or antagonists of present peptides (Greenwood, et al., J. Mol. Biol. 206:821-827, 1991).

There were only thirteen unique DNA sequences (out of a total of thirty) from B. subtilis spore-binding phage found. The frequency with which a particular sequence is found may directly reflect the tightness of binding of the encoded peptide. Although the sequences at positions five through seven are not identical, there is a clear preference for certain amino acids. Nearly one-third of all residues in positions 5, 6 and 7 are prolines (12/39), 31% are positively charged (5/39 Arg and 4/39 Lys), and the rest are hydrophobic or hydroxyl-containing. At position five, there is a strong preference for proline (6/13). Thus, it appears that these peptides bind to the same receptor on the spore coat.

(b) Determination of the 12-Mer Peptides

The biopanning experiment described above was repeated using a library containing larger 12-mer peptides shown in Table 2.

TABLE 2

| | |
|---|---|
| 1. Asn His Phe Leu Lys Ser Gln Pro Gly Val Val Thr | (SEQ ID No. 80) |
| 2. Asn His Phe Leu Asn Arg Pro Ala Gln Ser Gln Val | (SEQ ID No. 81) |
| 3. Asn His Phe Leu Pro Pro Lys Met Gly Pro Thr Asp | (SEQ ID No. 82) |
| 4. Asn His Phe Leu Pro Glu Pro Arg Leu Val Met Pro | (SEQ ID No. 83) |
| 5. Asn His Phe Leu Ala Pro Gln Pro Pro Val Lys Pro | (SEQ ID No. 84) |
| 6. Asn His Phe Leu Met Pro Asn Pro Leu Leu Ala Met | (SEQ ID No. 84) |
| 7. Asn His Phe Leu Ile Pro Pro Glu Pro Leu Arg Glu | (SEQ ID No. 85) |
| 8. Asn His Phe Leu Pro Leu Asn Pro Pro Ala Pro Ser | (SEQ ID No. 86) |

When the 12-mer peptides were compared with the 7-mer peptides, it appeared that no improvement occurred as a result of using the longer peptides.

(c) Determination of Binding Activities of 12-Mer Peptides

Selected peptides were analyzed for tight binding to the spore. Two peptides were synthesized initially: NHFLPKVGGGC (SEQ ID No. 16) and LFNKHVPGGGC (SEQ ID No. 17). The first has the amino-terminal sequence of peptide #4 plus a Gly₃ linker and a carboxy-terminal Cys. The second has a radomized sequence using the amino acids of peptide #4 plus the Gly₃ linker and carboxy terminal Cys. The goal was to label these peptides at the carboxy-terminus with phycoerythrin and examine binding of test and control peptides by fluorescence microscopy and FACS sorting.

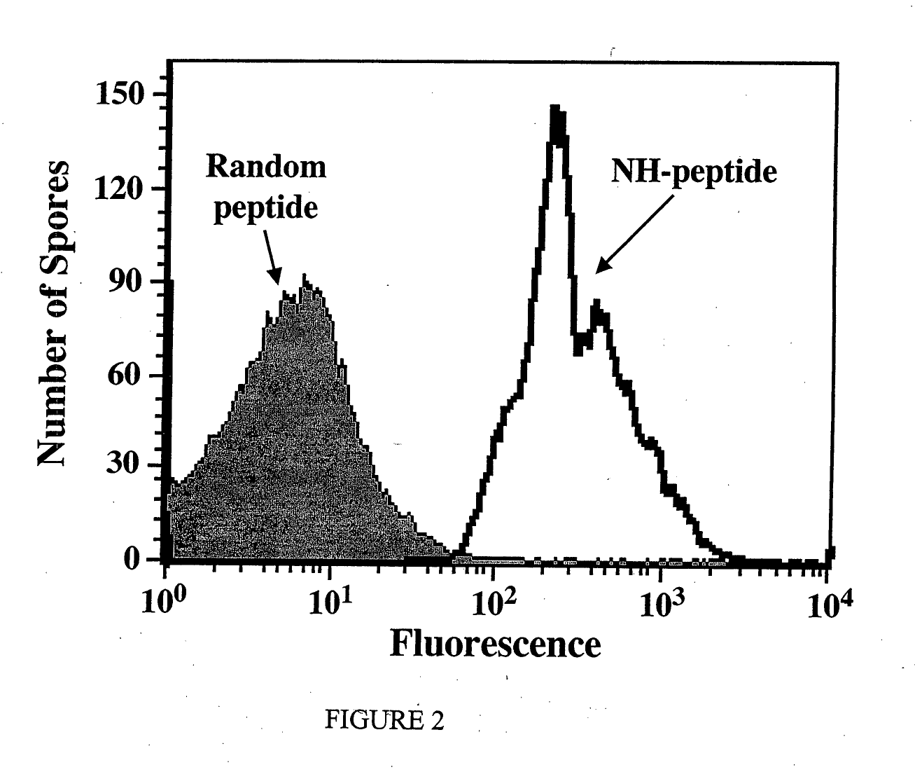

Initially, peptide-phycoerythrin conjugates were used for FACS. The advantage is that the conjugates are multivalent and the fluorescence characteristics are well suited for FACS. In some instances, peptides are first being reduced with tris(2-carboxyethyl)phosphine (TCEP) before conjugating with the phycoerythrin. Labeling with smaller fluorochromes such as monovalent 5-iodoacetamido-fluorescein is being used as an alternative. FIG. 2 depicts a representative FACS analysis of the binding of an NH-peptide-phycoerythrin conjugate to B. subtilis 168 spores. Briefly, Spores (10⁷) were mixed with an NH-peptide-phycoerythrin conjugate or a random 7-mer peptide-phycoerythrin conjugate (both at 3 μM and labeled at the same density) in 20 μl for 20 min. at room temperature. Spores were washed three times with 200 μl of TBST and 20,000 spores were analyzed. The fluorescence associated with sores treated with the random 7-mer peptide-phycoerythrin conjugate is no greater than that of untreated spores. The multiple fluorescent peaks with the NH-peptide are due to a mixture of single spores (the largest peak) and small spore aggregates (2 3, etc.).

In order to identify the receptor that interacts with the peptide, biotin-containing cross-linking agent that has been attached to a tight-binding peptide. Cross linkers examined included sulfosuccinimidyl-2-[6-(biotinamino)-2-(p-azidobenzamido)-hexanoamido]ethyl-1′c3′-dithiopropionate)sulfo-SBED). The molecule contains three different functional groups or arms. One arm consists of a biotin handle that can be used for purification using immobilized avidin. Another arm includes a sulfo-NHS (N-hydroxy-succinimido) ester that provides amino coupling capablility. When mixed with a tight-binding peptide, NHFLPKV plus GGGC (SEQ ID No. 99) extension, the cross linker is covalently coupled to the peptide through the ε-amino group of carboxy-terminal lysine residue, with the release of N-hydroxy-succinimide. To assure coupling only through the ε-amino group of the lysine, the amino terminus of the peptide (i.e., the α-amino group of asparagine) is temporarily protected. The third arm contains a photosensitive phenyl azide that can be activated by exposure to UV light at wave lengths greater than 300 nm. The activated phenyl azide reacts with nucleophiles, especially amines, in the target molecule.

Once the peptide-cross-linker conjugate was prepared, it was mixed with spores for 10 minutes in the dark to allow peptide-receptor interaction. The complexes were exposed to UV (365 nm) light for 15 minutes at 0° C. to allow cross-linking to the receptor. The spores were then collected by centrifugation, resuspended in SDS-PAGE loading dye (4% SDS, 10% β-mercaptoethanol, 1 mM dithiothreitol, 125 nM Tris-HCl (pH 6.8), 10% glycerol and 0.05% bromophenol blue) and boiled for 8 minutes to solubilized spore coat proteins (including receptor) and to reduce the disulfide bond that attaches the peptide to the cross-linking agent. Intact spores were removed by centrifugation. The supernatant containing solubilized proteins was dialyzed (MW cutoff: 2000 Da) against phosphate-buffered saline (PBS). The sample was passed over a monomer avidin column and washed with PBS to remove proteins lacking a biotin-containing cross-link. The bound protein/receptor was eluted with PBS containing 2 mM biotin. The fraction containing eluted protein (measured by OD₂₈₀) was dialyzed against H₂O and analyzed by SDS-PAGE. If one or more proteins were detected, they were analyzed by sequencing their amino terminus.

### EXAMPLE 3

### Identification of B. anthracis-Specific Peptides

(a) Biopanning of B. anthracis-Specific Peptides with Avirulent ΔAmes Strain of B. anthracis

Biopanning with the heptamer phage display library was used to identify tight-binding peptides on the surface of B. anthracis spores. The spores were prepared from the avirulent delta-Ames strain of the organism (lacking the toxin-encoding plasmid pOX1) and were sterilized by gamma-irradiation by Diagnostics Systems Division of the U.S. Army Medical Research Institute of Infectious Disease, Fort Detrick, Md.

Four rounds of biopanning were performed in the manner described above. The genomic DNA of amplified elutes from each round were sequenced directly and genomic DNA of 27 single plaques from the fourth round amplified elute were also sequenced. In the fourth round of biopanning, the defining decrease in titer of the supernatant and the increase in the titer of the elutate indicated the selection of tight-binding phages. The DNA sequences of the amplified eluates from biopanning rounds one through three did not show a change in the randomized 21 bp region. However, the DNA sequence of the amplified eluate from round 4 revealed a strong high-intensity shift and indicated a predominant DNA sequence. Reading the most intense bands in the 7-mer peptide encoding region gave the sequence TSQNVRT (SEQ ID No. 40).

The DNA sequences from single plaques of the fourth round of biopanning are summarized in Table 2. Thirteen of the 27 sequences were the same as the dominant sequence found in the amplified eluate. Two other closely related sequences TYPIPIR (SEQ ID No. 41) and TYPIPFR (SEQ ID NO. 42) were represented three times each. Another sequence TYPVPHR (SEQ ID No. 43) similar to the previous two sequences was found once. The three last sequences define a tight binding sequence of the consensus formula TYPX₁PX₂R (SEQ ID No. 119) wherein X defined hydrophobic residues and wherein the preferred X₁ is valine (V) or isoleucine (1) and preferred X₂ is isoleucine (I), Phenylalanine (F) or Histidine (H). A listing of the sequences is shown in Table 3. It has been shown that the first four amino acids of the most common sequence (TSQN) (SEQ ID 44) is present in domain 3 of the B. anthracis protective antigen.

Sequences 4, 5, 6 and 9 are preferred sequences for binding the spore coat.

TABLE 3

Nucleotide and Amino Acid Sequences from
B. anthracis Spore Binding Phage

| | | |
|---|---|---|
| Isolate | | |
| 1 (1) | AAT AGT GTT ACT CTT GAG CCG | (SEQ ID No. 60) |
| | Asn Ser Val Thr Leu Glu Pro | (SEQ ID No. 50) |
| 2 (1) | AAG CCG AGG CAG CCG GGT TTG | (SEQ ID No. 61) |
| | Lys Pro Arg Gln Pro Gly Leu | (SEQ ID No. 51) |
| 3 (1) | TCT ACT CCG GCG TGG CTG TCG | (SEQ ID No. 62) |
| | Ser Thr Pro Ala Trp Leu Ser | (SEQ ID No. 52) |
| 4 (13) | ACT AGT CAG AAT GTG CGG ACG | (SEQ ID No. 63) |
| | Thr Ser Gln Asn Val Arg Thr | (SEQ ID No. 40) |
| 5 (3) | ACT TAT CCT ATT CCG AAT CGT | (SEQ ID No. 64) |
| | Thr Tyr Pro Ile Pro Ile Arg | (SEQ ID No. 41) |
| 6 (3) | ACT TAT CCT AAT CCG TTT CGT | (SEQ ID No. 65) |
| | Thr Tyr Pro Ile Pro Phe Arg | (SEQ ID No. 42) |
| 7 (1) | TCT TAT CCT CAT GGT CAG ATT | (SEQ ID No. 66) |
| | Ser Tyr Pro His Gly Gln Ile | (SEQ ID No. 53) |
| 8 (1) | TTT ACT GGG ACT CTT AAT CCT | (SEQ ID No. 67) |
| | Phe Thr Gly Thr Leu Asn Pro | (SEQ ID No. 54) |
| 9 (1) | ACT TAT CCG GTG CCG CAT CCG | (SEQ ID No. 68) |
| | Thr Tyr Pro Val Pro His Arg | (SEQ ID No. 43) |
| 10 (1) | CGG ACT CCT TCG CTT CCT AGT | (SEQ ID No. 69) |
| | Arg Thr Pro Ser Leu Ser Pro | (SEQ ID No. 55) |
| 11 (1) | TTT AGT GTT CCT CGT ATG CCG | (SEQ ID No. 70) |
| | Phe Ser Val Pro Arg Met Pro | (SEQ ID No. 56) |

The number in ( ) refers to the number of phage containing the sequence.

(b) Biopanning and Characterization of B. anthracis-Specific Peptides with Avirulent Sterne and Delta-Ames Strain of B. anthracis

(i) Materials and Methods

Bacterial strains and spores. The Bacillus strains used in this study and their sources were as follows: the Sterne and delta-Ames strains of B. anthracis, B. cereus T, B. thuringiensis subsp. kurstaki, B. thuringiensis B8, and B. globigii (also called B. atrophaeus and B. subtilis variety niger) were from John Ezzell, U.S. Army Medical Research Institute of Infectious Diseases, Fort Detrick, Md.; B. thuringiensis Al Hakum, B. thuringiensis USDA HD-571, B. cereus 3A (also FRI-41), B. cereus F1-15 (also FRI-43), B. cereus D17 (also FRI-13), and B. cereus S2-8 (also FRI-42) were from Paul Jackson, Los Alamos National Laboratory, Los Alamos, N. Mex.; B. subtilis (trpC2) 1A700 (originally designated 168), B. amyloliquefaciens 10A1 (originally H), B. licheniformis 5A36 (originally ATCC 14580), and B. pumilus 8A3 (originally ATCC 7061) were from the Bacillus Genetic Stock Center, Ohio State University, Columbus; and B. cereus ATCC 4342, B. mycoides ATCC 10206, and B. megaterium ATCC 14581 were from the American Type Culture Collection, Manassas, Va. Spores were produced by cells grown in liquid Difco sporulation medium [Nicholson et al., Sporulation, germination and outgrowth, p. 391-450. In C. R. Harwood and S. M. Cutting (ed.), Molecular biological methods for Bacillus. John Wiley & Sons, Ltd., West Sussex 1990] at 37° C. for 48 to 72 h with shaking (except for B. pumilis, which was grown on solid medium at 30° C. Spores were purified by sedimentation through a Renografin step gradient as previously described [Henriques et al., J. Bacteriol. 177:3394-3406 (1995)] and were quantitated microscopically using a Petroff-Hausser counting chamber. In the biopanning experiment with B. anthracii ΔAmes spores, the spores were killed by gamma irradiation before use (an initial precautionary measure). In all other biopanning and spore-binding experiments (including those with ΔAmes spores), unirradiated spores were used. Gamma irradiation of spores did not appear to affect peptide binding.

Screening the phase display peptide library The New England Biolabs (NEB) Ph.D.-7 Phage Display Peptide Library was biopanned for spore-binding phages, and these phages were analyzed as described previously.

Peptide synthesis and fluorochrome conjugation Peptides were chemically synthesized and purified by high performance liquid chromatography (University of Alabama at Birmingham Peptide Synthesis Core Facility). Peptide molecules were attached to R-phycoerythrin (PE; Prozyme) by using the heterobifunctional cross-linker sulfosuccinimidyl-4-(N-maleimidomethyl)cyclohexane-1-carboxylate.

FACS analysis Spores (10⁷) were mixed with a peptide-PE conjugate (at a concentration indicated in the text) in 20 μl of phosphate buffered saline (PBS) and incubated at room temperature for 60 min to ensure complete binding. Unbound conjugate molecules were removed by washing spores three times in 200-F1 volumes of PBS-0.5% Tween-20; after each wash, spores were collected by centrifugation at 820×g and 4° C. for 5 min. Spore-conjugate complexes were resuspended in 200 μl PBS, and fluorescence was measured by FACS analysis with a FACS Calibur instrument and analyzed with CellQuest Pro software (Becton Dickinson Biosciences). Spore structure was unaffected by this assay as judged by microscopic examination.

Fluorescence microscopy Spores (10⁸) were mixed with a peptide-PE conjugate, incubated, and washed essentially as described for the FACS assay. The spore pellet was resuspended in a drop of Fluoromount-G (Electron Microscopy Sciences, Fort Washington, Pa.), and a sample was examined under a Nikon Eclipse E600 microscope with a Y-FL epifluorescence attachment. Fluorescence micrographs were taken with a Spot charge-coupled device camera (Diagnostic Instruments Inc., Sterling Heights, Mich.), using a 5-s exposure time and a gain of 8.

(b) Biopanning a Phage Display Library for Peptides that Bind B. anthracis Spores.

To identify peptides that bind to spores of B. anthracis, the NEB Ph.D.-7 Phage Display Peptide Library was screened for spore-binding phages. In the Ph.D.-7 library, random 7-mer peptides were displayed on the surface of the filamentous coliphage M13 as fusions to the surface-exposed amino terminus of the minor coat protein PIII. Each phage contained five copies of the same peptide-PIII fusion, which was encoded by the phage gene III containing a random 21-base insert. The phage display library contained 2×10⁹ independent clones. In two separate experiments, the phage display library was biopanned against purified spores produced by either the delta-Ames or Sterne strain of B. anthracis. The ΔAmes (pXO1⁻) and Sterne (pXO2⁻) strains are avirulent due to the absence of a plasmid necessary to produce anthrax toxins or the capsule of the vegetative cell, respectively. For each biopanning experiment, spores and phages were mixed to allow binding, spore-phage complexes were collected by centrifugation and washed ten times, phages were eluted from the complexes, and the eluted phages were amplified by infecting E. coli cells (under conditions similar to those recommended by NEB). The amplified phages were used for a second round of biopanning; in total, four rounds of biopanning were performed, after which the eluted phages (each displaying a putative spore-binding peptide) were plated to obtain single plaques. These plaques (27 and 35 with delta-Ames and Sterne spores, respectively) were used to prepare phage stocks, from which genomic DNA was purified, and the 21-base insert in gene III of each phage was sequenced. The amino acid sequences encoded by the inserts revealed putative spore-binding 7-mer peptides.

Based on related sequences, the peptides were grouped into several families, each defined by a unique consensus sequence. However, only one peptide family was found in both biopanning experiments (i.e., with delta-Ames and Sterne spores). This family, with the consensus sequence TYPX₃PX₄R (SEQ ID No. 120) wherein X defined hydrophobic residues and wherein the preferred X₃ is valine (V) isoleucine (1), proline (P) or leucine (L) and preferred X₄ is isoleucine (I), phenylalanine (F), histidine (H) or threonine (T), was the largest in terms of number of phages (19 of 62) and unique peptide sequences (Table 4).

TABLE 4

| | | |
|---|---|---|
| 1 | ACT TAT CCG GTG CCG CAT CGG | (SEQ ID No. 68) |
| | Thr Tyr Pro Val Pro His Arg | (SEQ ID No. 43) |
| 2 | ACT TAT CCT ATT CCG AAT CGT | (SEQ ID No. 64) |
| | Thr Tyr Pro Ile Pro Ile Arg | (SEQ ID No. 41) |
| 3 | ACT TAT CCT AAT CCG TTT CGT | (SEQ ID No. 65) |
| | Thr Tyr Pro Ile Pro Phe Arg | (SEQ ID No. 42) |
| 4 | ACG TAT CCG CTG CCG ATT CCG | (SEQ ID No. 131) |
| | Thr Tyr Pro Leu Pro Ile Arg | (SEQ ID No. 121) |
| 5 | ACG TAT CCG CTG CCT ATT AGG | (SEQ ID No. 132) |
| | Thr Tyr Pro Leu Pro Ile Arg | (SEQ ID No. 122) |
| 6 | ACT TAT CCG CCG CCG ACT CTT | (SEQ ID No. 133) |
| | Thr Tyr Pro Pro Pro Ile Arg | (SEQ ID No. 123) |

The consensus sequence TYPX₃PX₄R (SEQ ID No. 120) is hereafter referred to as TYP. Often, more than one phage clone displayed a particular TYP peptide, and this peptide was encoded by the same nucleotide sequence. In one case, the peptide sequence (i.e., TYPLPIR SEQ ID No. 122) was encoded by two different nucleotide sequences, as permitted by the degeneracy of the genetic code. Although the TYP consensus sequence was variable at positions 4 and 6, the residues at these positions were typically similar. For example, Leu, Ile, or Val occupied position 4 in all but one unique peptide sequence.

(c) Analysis of Spore Binding by TYP Peptides

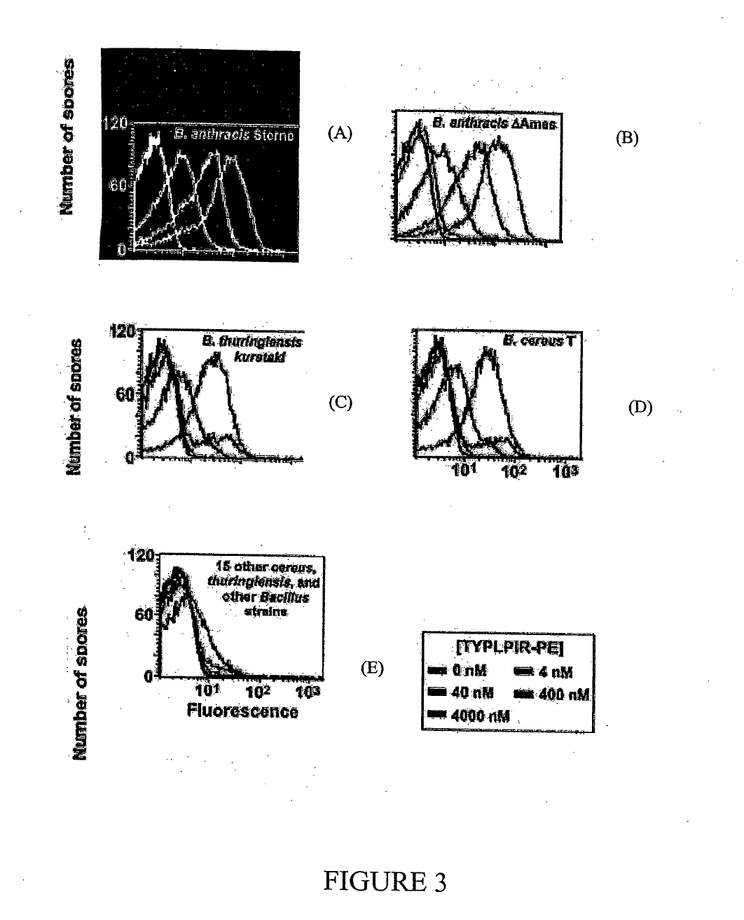

To confirm and analyze the binding of TYP peptides to spores, we employed a FACS assay. This assay required the attachment of a fluorochrome to a test peptide prior to spore binding and analysis of spore-peptide complexes. To this end, we chemically synthesized a representative TYP peptide with the sequence TYPLPIRGGGC (SEQ ID No. 124); the GGGC extension was included as a carboxy-terminal linker for fluorochrome attachment. Approximately ten peptide molecules were then attached (using a cross-linker) through their terminal cysteine residues to the ε-amino groups of dispersed lysine residues on one molecule of PE, a 240-kDa highly fluorescent protein. Peptide binding to B. anthracis (Sterne and ΔAmes) spores was then measured by incubating spores with from 4 to 4,000 nM peptide-PE conjugate, removing unbound conjugate by washing, and analyzing spore-peptide complexes by FACS. The results showed essentially identical, concentration-dependent binding of the peptide-PE conjugate to spores of the Sterne and delta-Ames strains (FIG. 3).

To examine the specificity of peptide binding, the binding of the TYPLPIRGGGC-PE conjugate to spores of 17 other Bacillus strains, including 6 strains of B. cereus (T, ATCC 4342, D17/FRI-13, 3A/FRI-41, S2-8/FRI-42, and F1-15/FRI-43), 4 strains of B. thuringiensis (subsp. kurstaki, B8, Al Hakum, and USDA HD-571), and one strain each of B. mycoides, B. pumilus, B. globigii, B. amyloliquefaciens, B. subtilis, B. licheniformis, and B. megaterium, was determined. These strains were all members of Bacillus Group 1 (of 5), within which B. anthracis, B. cereus, B, thuringiensis, and B. mycoides comprise the closely related B. cereus group. Seven of these strains—i.e., B. thuringiensis strains Al Hakum and USDA HD-57 and all B. cereus strains except T—are human pathogens and nearest neighbors to B. anthracis as determined by amplified fragment length polymorphism analysis [Radnedge et al., Appl. Environ. Microbiol. 69:2755-2764 (2003)]. The binding assays showed that the peptide-PE conjugate did not bind to 15 of the other Bacillus strains (FIG. 3) (minimal binding at a conjugate concentration of 4,000 mM was due to non-specific entrapment). Peptide binding was detected for spores of B. cereus T and B. thuringiensis subsp. kurstaki, but this binding was weaker (or less extensive) than that observed with B. anthracis Sterne and ΔAmes spores. These results indicated a high degree of specificity in TYPLPIR binding to spores but revealed that binding was not absolutely restricted to B. anthracis spores.

To control for nonspecific binding in each experiment shown in FIG. 3, several dissimilar 11-mer peptides (for example, HWHHHGHGGGC (SEQ ID No. 125) and ILPRPYTGGGC (SEQ ID No. 126), the latter being a scrambled version of a TYP peptide) were attached to PE as described above. These conjugates were tested for spore binding. No significant binding was detected. In a related control experiment, we showed that binding of the TYPLPIRGGGC-PE conjugate to B. anthracis spores was not inhibited by inclusion of bovine serum albumin at 10 mg/ml in the binding and wash buffers. Furthermore, it was found that the TYPLPIRGGGC-PE conjugate did not bind to vegetative cells of the Sterne and ΔAmes strains (data not shown).

(d) Enhanced Spore Binding by ATYPLPIR (SEQ ID No. 127)

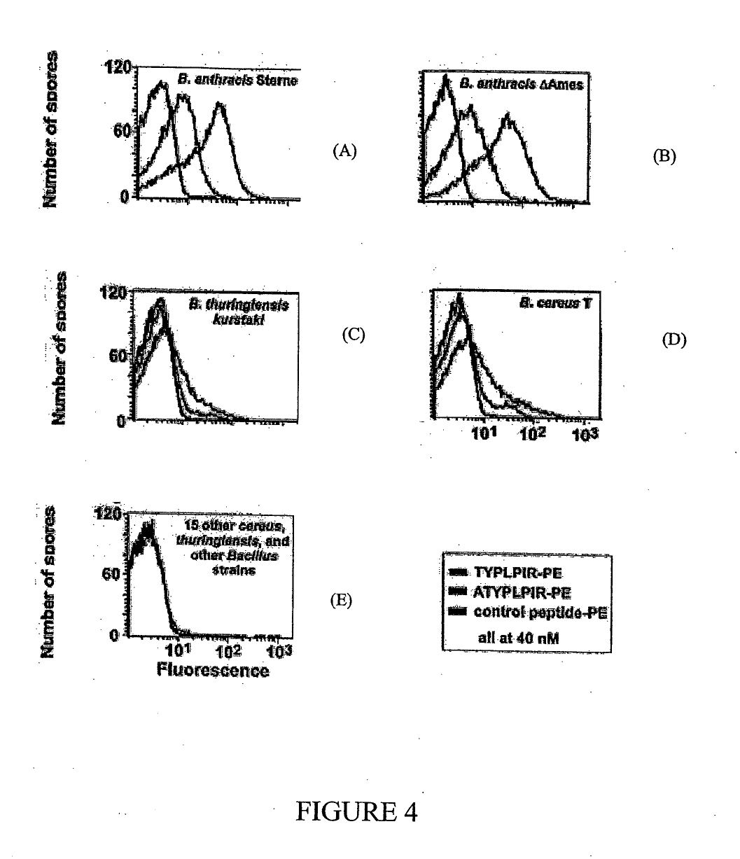

A family of 7-mer and 12-mer peptides that selectively bound B. subtilis spores was identified. This family contained a five-residue consensus sequence that permitted spore binding only when present at the amino terminus of a peptide or protein. To determine if TYP also required a free amino terminus for spore binding, a peptide with the sequence ATYPLPIRGGGC (SEQ ID No. 128), which is a representative of the general formula ATYP (i.e., ATYPX₃PX₄RGGGC (SEQ ID No. 132) was synthesized and attached it to PE as described above. The spore-binding ability of this conjugate was compared to that of TYPLPIRGGGC-PE, with both conjugates used at a concentration of 40 nM (FIG. 4). The results showed clearly that the TYPLPIR (SEQ ID No. 122) sequence did not require a free amino terminus to bind spores. In fact, the addition of an Ala residue permitted nearly 10-fold-enhanced binding to both Sterne and delta-Ames spores. Binding to B. thuringiensis subsp. kurstaki and B. cereus T spores was only slightly enhanced by the Ala addition, while this modification still did not permit detectable binding to spores of all other species examined. These experiments provided us with an improved ligand for B. anthracis spores, namely ATYPLPIR (SEQ ID No. 127), and suggested that even better peptide ligands can be produced by additional modifications. Presumably, ATYP (i.e., ATYPX₃PX₄R (SEQ ID No. 129) and TYP peptides bind to the same spore receptor, although this remains to be confirmed.

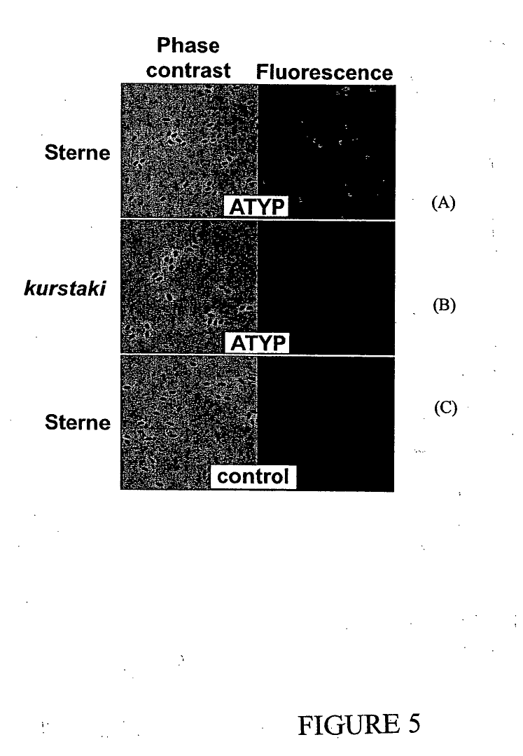

To demonstrate directly that the ATYPLPIR (SEQ ID No. 122) peptide was binding to the spore surface, the binding of the ATYPLPIRGGGC-PE conjugate to B. anthracis spores was examined by fluorescence microscopy. The results showed that when binding occurred at a conjugate concentration of 400 nM and unbound conjugate was removed by washing, every Sterne or delta-Ames spore was completely encircled by fluorescent ligand (data not shown). At this concentration of conjugate, low-level but detectable binding to spores of B. thuringiensis subsp. kurstaki and B. cereus T (but not other strains) was observed. In an attempt to identify conditions under which only spores of B. anthracis would bind enough conjugate to be detectable by fluorescence microscopy, it was found that at a conjugate concentration of 40 nM, Sterne and ΔAmes spores were readily detectable, although with somewhat uneven fluorescence, while spores of the other 17 Bacillus strains examined in this study were essentially nonfluorescent (FIG. 5). In addition, we used several control peptide-PE conjugates to confirm that fluorescent labeling of spores required the ATYPLPIR (SEQ ID No. 122) sequence (FIG. 5 and data not shown). The reason for the uneven fluorescence observed with B. anthracis spores at 40 nM ATYPLPIRGGGC-PE is not known, but it appears to be unrelated to spore damage (e.g., loss of the outer spore layer).

(e) Use of Two Peptides for Unambiguous Spore Identification

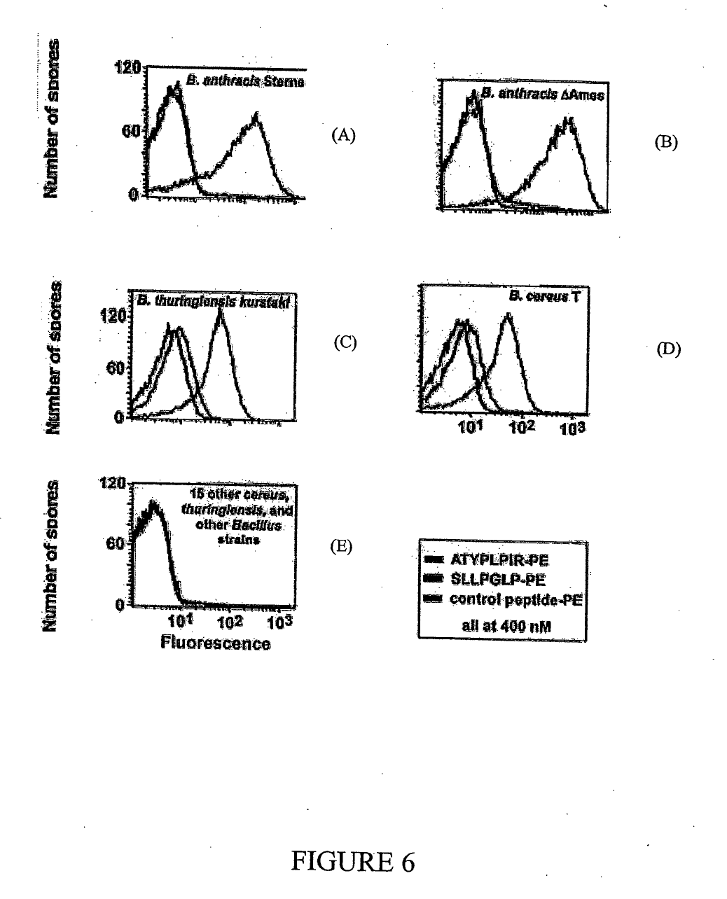

In yet another biopanning experiment, the Ph.D.-7 Phage Display Peptide Library was screened for peptides that bind to spores of an uncharacterized Bacillus strain (probably a strain of B. cereus or B. thuringiensis) that was isolated from an environmental sample (data not shown). These peptides revealed a single consensus sequence, SLLPGL (SEQ ID No. 130), which was subsequently shown to bind to spores (but not to vegetative cells) of only two strains in our collection, B. thuringiensis subsp. kurstaki and B. cereus T. The fact that the SLLPGL (SEQ ID No. 130) peptides bind well to spores of B. thuringiensis subsp. kurstaki and B. cereus T, the only non-B. anthracis spores that were found to bind TYP peptides, suggested that SLLPGL (SEQ ID No. 130) peptides could be used in tandem with TYP or ATYP peptides to unambiguously identify B. anthracis spores. To demonstrate this application, an SLLPGLPGGGC (SEQ ID No. 131)-PE conjugate that was equivalent to the previously described ATYPLPIRGGGC-PE conjugate was prepared. The abilities of the SLLPGL (SEQ ID No. 130) and ATYP conjugates (at 400 nM concentrations) to bind to spores of the 19 Bacillus strains used in this study were compared by FACS assay (FIG. 6). The binding pattern for spores of both strains of B. anthracis was unique, with extensive binding by the ATYP conjugate and no binding by the SLLPGL (SEQ ID No. 130) conjugate. In clear contrast, the binding pattern for spores of B. thuringiensis subsp. kurstaki and B. cereus T was essentially reversed, with extensive binding by the SLLPGL (SEQ ID No. 130) conjugate and limited binding by the ATYP conjugate. No peptide-conjugate binding was observed with the other 15 spore types. Thus, the SLLPGL (SEQ ID No. 130) and ATYP conjugates can be used to clearly distinguish between spores of B. anthracis and the other Bacillus species examined.

### EXAMPLE 4

### Identification of B. cereus-Specific Peptides

Studies with B cereus T were undertaken using methods described above. After the fourth round of biopanning, individual phages were cloned. Twenty-two phage cones were picked, the genomic DNA prepared from each, and the DNA coding regions sequenced. The results revealed 8 unique DNA sequences. (See Table 5.) A heptapeptide VTSRGNV (SEQ ID No. 111) having tight-binding properties was identified. This sequence emerged from the pooled genome sequence of amplified phage following the third round of biopanning. The following unique DNA sequences were identified.

TABLE 5
B. cereus T spore tight-binding peptides:

| | | |
|---|---|---|
| 1. (2) | ACG CAT CGT TTG CCT TCT CGG | (SEQ ID No. 101) |
| | Thr His Arg Leu Pro Ser Arg | (SEQ ID No. 110) |
| 2. (13) | GTT ACT AGT AGG GGG AAT GTT | (SEQ ID No. 102) |
| | Val Thr Ser Arg Gly Asn Val | (SEQ ID No. 111) |
| 3. | AAG CTG TGG GTG ATT CCT CAG | (SEQ ID No. 103) |
| | Lys Leu Trp Val Ile Pro Gln | (SEQ ID No. 112) |
| 4. | TAT TCG CCT CCT CAT AGG CAT | (SEQ ID No. 104) |
| | Tyr Ser Pro Leu His Arg His | (SEQ ID No. 113) |
| 5. | TCG TAT CTT CCG TAT TTT GAT | (SEQ ID No. 105) |
| | Ser Tyr Pro Pro Tyr Phe Asp | (SEQ ID No. 114) |
| 6. (2) | CTT TTG TCG CCT CTG CAT CGT | (SEQ ID No. 106) |
| | Leu Leu Ser Pro Leu His Arg | (SEQ ID No. 115) |
| 7. | TTT GAT TCT CCG CTT CGT CGG | (SEQ ID No. 107) |
| | Phe Asp Ser Pro Leu Arg Arg | (SEQ ID No. 116) |
| 8. | TGG TCG CCG CTG CAT AAG CAT | (SEQ ID No. 108) |
| | Trp Ser Pro Leu His Lys His | (SEQ ID No. 117) |

One DNA sequence (SEQ ID No. 102), found in 13 of the 22 phage, encoded the previously identified tight-binding peptide VTSRGNV (SEQ ID No. 111). A second sequence was obtained from an inspection of the remaining 7 unique phage DNA sequences. Four of these sequences (4, 6, 7 and 8) contained all or most of the closely related sequence SPL(H or R)(R or K)H (SEQ ID No. 100). Such results are highly suggestive of a true tight-binding peptide sequence.

A competitive biopanning study was performed using phage displaying unique peptide sequence 8 (WSPLHKH) (SEQ ID No. 117) in this study. 10¹⁰ phage from a random phage library and 10⁷ phage displaying sequence #8 were mixed together, and one round of biopanning was performed using spores of B. cerues T. The eluted phage were plaque-purified and genomic DNA was sequenced for twenty phage. Six of the twenty phage contained the sequences of isolate #8. Thus, there was a 300-fold enrichment of this phage, indicating tight binding.

In efforts to find additional tight-binding peptides the wash buffer or wash conditions have been systematically modified. One alteration is the use of either 0.5% or 0.01% Tween 20 and either 3 or 10 washes. These conditions were used in biopanning for B subtilis, wherein number of washes were reduced from 10 to 3. After four rounds of biopanning, an amplified eluted phage pool was sequenced. The results indicate that by changing certain parameters, it is possible to detect new tight binders for some spore species.

It was found that it was possible to precipitate the amplified phage for 30 minutes instead of overnight. Using the abbreviated method, it was possible to omit all titering of phage between the rounds. Using this method, an approximate concentration of amplified phage (1.75×10¹³ pfu/ml) is assumed. Omitting these steps allows a four-round biopanning experiment to be completed in two 12-hour days without affecting results.

The preferred embodiments of the compounds and methods of the present invention are intended to be illustrative and not limiting. Modifications and variations can be made by persons skilled in the art in light of the above teachings. It is also conceivable to one skilled in the art that the present invention can be used for other purposes of measuring the acetone level in a gas sample, e.g. for monitoring air quality. Therefore, it should be understood that changes may be made in the particular embodiments disclosed which are within the scope of what is described as defined by the appended claims.

What is claimed is:

1. An isolated peptide which binds to a B. anthracis spore, said peptide comprising an amino acid sequence selected from the formula of ATYPX₃PX₄R (SEQ ID NO: 129) or ATYPX₃PX₄RGGGC (SEQ ID NO:132), wherein said X₃ is Ile, Val, Leu or Pro; X₄ is Ile, Phe, His, or Thr.

2. The isolated peptide of claim 1, wherein said X₃ is Leu or Val, X₄ is Ile, or Thr.

3. The isolated peptide of claim 2, wherein said X₃ is Leu and X₄ is Ile.

4. An isolated peptide which binds to a B. anthracis spore, said peptide comprising an amino acid sequence of SEQ ID NO: 127, or SEQ ID NO: 128.

5. An isolated peptide which binds to a B. anthracis spore, said peptide comprising an amino acid sequence selected from the formula of TYPX₁PX₂R (SEQ ID NO: 119), wherein said X₁ is Ile, Val, Leu or Pro; X₂ is Ile, Phe, His, or Thr.

6. The isolated peptide of claim 5, wherein said X₁ is Val or Leu X₂ is Ile or Phe.

7. An isolated peptide which binds to a B. anthracis spore, said peptide comprising an amino acid sequence of SEQ ID NO: 50, SEQ ID NO: 51, SEQ ID NO: 52, SEQ ID NO: 40, SEQ ID NO: 41, SEQ ID NO: 42, SEQ ID NO: 53, SEQ ID NO: 54, SEQ ID NO: 43, SEQ ID NO: 55, SEQ ID NO: 56, SEQ ID NO: 121, SEQ ID NO: 122, or SEQ D NO: 123.

8. The isolated peptide of claim 7, wherein said peptide has an amino acid sequence which is selected from the group consisting of: SEQ ID NO: 40, SEQ ID NO: 41, SEQ ID NO: 42, SEQ ID NO: 43, SEQ ID NO: 121, SEQ ID NO: 122, and SEQ ID NO: 123.

9. The isolated peptide of claim 7, wherein said peptide has the amino acid sequence of SEQ ID NO: 40.

10. The isolated peptide of claim 7, wherein said peptide has the amino acid sequence of SEQ ID NO: 41.

11. The isolated peptide of claim 7, wherein said peptide has the amino acid sequence of SEQ ID NO: 42.

12. The isolated peptide of claim 7, wherein said peptide has the amino acid sequence of SEQ ID NO: 43.

13. The isolated peptide of claim 7, wherein said peptide has the amino acid sequence of SEQ ID NO: 121.

14. The isolated peptide of claim 7, wherein said peptide has the amino acid sequence of SEQ ID NO: 122.

15. The isolated peptide of claim 7, wherein said peptide has the amino acid sequence of SEQ ID NO: 123.

16. A method for detecting a B. anthracis spore in a sample, said method comprising:
(a) contacting said sample to a peptide which has an amino acid sequence of SEQ ID NO. 127 or SEQ ID NO. 128;
(b) determining the presence of said B. anthracis spores in said sample.

17. The method of claim 16, wherein said determining the presence of B. anthracis spores in said sample is performed using fluorescence-activated cell sorting (FACS).

18. The method of claim 16, wherein said determining the presence of B. anthracis spores in said sample is performed using fluorescence microscopy.

19. A method of detecting a B. anthracis spore in a sample, said method comprising:
(a) contacting said sample to a peptide which comprises an amino acid sequence of SEQ ID NO: 40, SEQ ID NO: 41, SEQ ID NO: 42, SEQ ID NO: 43, SEQ ID NO: 121, SEQ ID NO: 122, or SEQ ID NO: 123,
(b) determining the presence of said B. anthracis spores in said sample.

20. The method of claim 19, wherein said determining the presence of B. anthracis spores in said sample is performed using fluorescence-activated cell sorting (FACS).

21. The method of claim 19, wherein said determining the presence of B. anthracis spores in said sample is performed using fluorescence microscopy.

22. A method of identifying a peptide which binds to a B. anthracis spore comprising the steps of:
a. mixing phage from a Phage Display library with said B. anthracis spores;
b. incubating the product of step (a) for sufficient time to allow the formation of phage-spore complexes;
c. obtaining said phage-spore complexes;
d. washing said phage-spore complexes repeatedly;
e. eluting the phage from said phage-spore complexes with elution buffer;
f. amplifying the eluted phage;
g. purifying individual clones;
h. amplifying purified clones, and then extracting genomic DNA from each preparation.

23. The method of claim 22, following the step (e), further comprising the step of neutralizing said eluate.

24. The method of claim 22, further comprising the step (i), determining the DNA sequence encoding peptides.

25. The method of claim 24, following the step (i), further comprising the step of testing binding activity of said peptides to said anthracis spores.

26. The method of claim 22, following the step (f), further comprising the step of repeating said steps (a)-(f) 2 to 5 rounds.

27. A Kit for detecting a B. anthracis spore comprising a peptide of claim 4.

28. The Kit of claim 27 further comprising a detective tag.

29. The Kit of claim 28, wherein said detective tag is fluorescent agent, phosphorescent agent, colorimetric agent or radioactive agent.

30. A Kit for detecting a B. anthracis spore comprising a peptide of claim 7.

31. The Kit of claim 30 further comprising a detective tag.

32. The Kit of claim 31, wherein said detective tag is fluorescent agent, phosphorescent agent, colorimetric agent or radioactive agent.
